(19) United States
(12) Patent Application Publication
(10) Pub. No.: US 2009/0318907 A1
(43) Pub. Date: Dec. 24, 2009

(54) GENERALIZED MODELING OF THE CORNEA

(76) Inventors: Josef F. Bille, Heidelberg (DE); Luis Antonio Ruiz, Bogota (CO); Frieder Loesel, Mannheim (DE)

Correspondence Address: NYDEGGER & ASSOCIATES, 348 OLIVE STREET, SAN DIEGO, CA, 92103, US

(21) Appl. No.: 12/143,600

(22) Filed: Jun. 20, 2008

(51) Int. Cl. A61B 18/20

(52) U.S. Cl. 606/5

(57) ABSTRACT

A system and method for simulating a corneal reconfiguration in response to laser surgery uses a computer-programmed, biomechanical generalized model. The generalized model has a plurality of elements; with each element being pre-programmed based on diagnostic corneal data obtained from images of respective individual collagen fibers in a cornea. Collectively these pre-programmed elements replicate biomechanical properties of the cornea. In use, designated biomechanical characteristics on a plurality of selected elements are minimized to simulate laser surgery in an actual cornea. A computer then measures the resultant reconfiguration of the cornea model to assess an actual cornea's response to laser surgery.

### FIELD OF THE INVENTION

The present invention pertains generally to computer models. More particularly, the present invention pertains to models for the cornea of an eye that can be used to predict a corneal response to a predetermined stimulus. The present invention is particularly, but not exclusively, useful as a biomechanical model for a cornea that is defined and based on data pertaining to individual collagen fibers in a cornea.

### BACKGROUND OF THE INVENTION

Computer modeling has proven to be a very helpful design tool for many technical endeavors. This is particularly so when complex structures are involved. And more so, when a response of the structure to changes in forces on the structure must be predicted with great accuracy. Such is the case with the cornea of an eye.

Anatomically, the cornea of an eye is a combination of several (i.e. five) different layers of tissue. Going in a direction from the anterior surface of the cornea toward its posterior surface, these layers are: the epithelium, Bowman's membrane, stroma, Descemet's membrane and the endothelium. Importantly, Bowman's membrane and the stroma structurally constitute more than ninety percent of the cornea, and both these tissues are made of collagen.

A collagen fiber is a fibrous protein that is abundantly found in the extracellular matrix, tendons and bones of animals. For purposes of modeling the cornea of an eye, they can be mathematically defined in terms of their elasticity, their viscosity, and their respective shape (i.e. length and orientation in the cornea). Further, within the cornea itself, collagen fibers can be classified by “type”. In general, this classification accounts for the fiber's length, as well as its cross linking bonds with other fibers. This classification also accounts for the density of fibers in a defined volume of tissue. Although more than one “type” of collagen fiber may be present in a given tissue (e.g. the stroma), the predominance of one “type” collagen fiber will give the tissue its basic characteristics. For example, collagen fibers in Bowman's membrane are classified as “type I” or “type III” fibers. On the other hand, collagen fibers in the stroma will be mostly “type V” and “type VI” fibers. In this example, “type III” fibers are shorter, have more cross linking bonds with other fibers, and are more densely arranged than are either “type V” or “type VI” fibers. Stated differently, with a higher number “type”, a collagen fiber will be longer, have less cross linking bonds with other fibers, and will be less densely arranged. Importantly, these differences can be quantified.

It is possible to image collagen fibers in the cornea. Specifically, it is known that by using well known second harmonic generation techniques, around one thousand images of a cornea can be obtained within about one minute. These images can then be used to ascertain the length and orientation of as many individual collagen fibers as are needed (e.g. tens of thousands and, possibly, millions). Also, changes in physical properties of the collagen fibers can be observed by taking images of collagen fibers under different pressure conditions in the eye. These observations can then be compared and used to attribute elastic and viscous properties to the particular fibers. The data thus collected for all fibers can then be used as input for a computer model.

As envisioned for the present invention, all of the data regarding collagen fibers that is collected as indicated above, can be used to define the constituents of a generalized model cornea. At this point it is important to note, there is no need to differentiate specific layers of the cornea (e.g. Bowman's membrane and the stroma). Instead, tissue distinctions within the cornea are accounted for by data acquired from images of individual collagen fibers, and their arrangements (i.e. their “type”). The generalized model can then be further defined with an anterior surface and a posterior surface using mathematical approximations. Thereafter, standard computer techniques can be employed to ascertain responses of the generalized model to selected stimuli.

In light of the above, it is an object of the present invention to provide a generalized biomechanical model of a cornea that is based on the physical characteristics of individual collagen fibers. Another object of the present invention is to provide a generalized biomechanical model of a cornea that comprises a substantially uninterrupted, essentially continuous, data presentation of corneal tissue attributes. Yet another object of the present invention is to provide a generalized model of a cornea that is easy to use and comparatively cost effective.

### SUMMARY OF THE INVENTION

In accordance with the present invention, a system and method for simulating the reshaping of a cornea requires a generalized model of a cornea and a computer that is electronically connected to the model. Specifically, the computer is connected with the model to selectively stimulate the model and to measure its response to the input stimulus. For the present invention, the model is based on diagnostic data obtained from collagen fibers in the cornea that is being modeled. Both the anterior surface of the model cornea and the posterior surface of the model cornea are based on mathematical approximations.

In detail, the diagnostic data that is used to create the generalized model cornea is taken from different images of the cornea, and is used to establish biomechanical characteristics for the model. As envisioned for the present invention, these images can be taken by any means known in the pertinent art, such as by second harmonic generation imaging. Further, these images are preferably generated under different pressure conditions. Consequently, individual collagen fibers in these images can be identified, classified and characterized under the influence of a pressure differential. Thus, not only can the length and orientation of individual collagen fibers be determined, their individual responses to the pressure differential can also be observed. This information is then collectively used, along with general characteristics that are attributed to the “type” of fiber, to establish elastic and viscous properties for specific elements in the model. Each element so established corresponds to an individual collagen fiber in the images.

As indicated above, mathematical approximations are used to define the surfaces for the model cornea. In particular, the anterior surface and the posterior surface for the cornea are modeled by considering an axis perpendicular to the surfaces and passing through respective apexes. The surfaces are further considered as having curvatures that are approximated by a respective conic section. In this case, the conic section for each surface is expressed as:

$$z ( x ) = \frac{1}{{e}^{2} - 1} [ \sqrt{{R}^{2} + {x}^{2} ( {e}^{2} - 1 )} - R ] .$$

For the above expression, “R” is the radius of curvature of a respective corneal surface, and “e” is the eccentricity of the cornea.

In its operation, the present invention requires use of a generalized model cornea that is programmed as described above. Specifically, the model cornea has its plurality of elements pre-programmed to respectively simulate biomechanical characteristics of individual collagen fibers in the cornea. The computer can then be used to stimulate the model. For this stimulation, the biomechanical characteristics on selected elements are minimized. Then, the cornea which is reshaped in response to the minimization, is measured and evaluated. Several iterations of this minimization, measuring and evaluation can be accomplished until the response is considered an indication of an accurate and precise outcome. An actual, surgical operation can then be performed, accordingly.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic representation of the interactive components of the present invention;

FIG. 2 is a perspective view of a cornea (model cornea);

FIG. 3 is a perspective representation of a plurality of lamellae of collagen fibers;

FIG. 4 is a representation of a plurality of individual (“type I, III”) collagen fibers, typical of tissue in Bowman's membrane of a cornea;

FIG. 5 is a representation of a plurality of individual (“type V, VI”) collagen fibers, typical of tissue in the stroma of a cornea;

FIG. 6 is a cross section view of a cornea as seen along the line 6-6 in FIG. 2 under different pressure conditions;

FIG. 7A shows a collagen fiber with a shape and orientation under a first pressure condition; and

FIG. 7B shows the collagen fiber of FIG. 7A under a second pressure condition.

### DESCRIPTION OF THE PREFERRED EMBODIMENTS

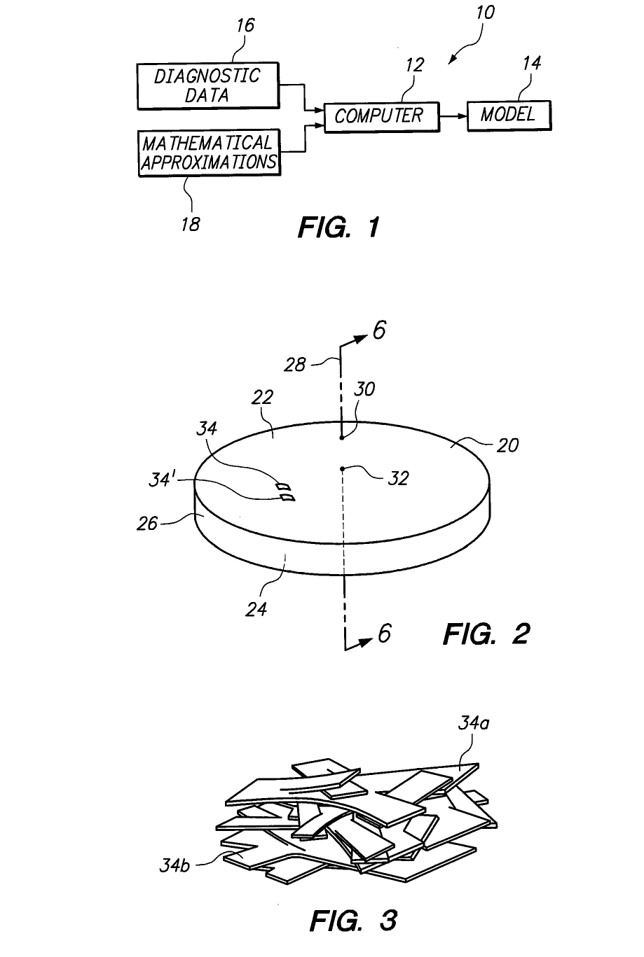

Referring initially to FIG. 1 a system in accordance with the present invention is shown schematically and is generally designated 10. In FIG. 1 it will be seen that the system 10 includes a computer 12 electronically connected to a model 14. Further, FIG. 1 indicates that diagnostic data 16 and mathematical approximations 18 are provided as input to the computer 12. The computer 12 will then use the diagnostic data 16 and the mathematical approximations 18 for the creation of the model 14. Thereafter, the computer 12 can use the model 14 for purposes of evaluating physical changes to a cornea 20 that may result in response to selected stimuli.

For purposes of the present invention, a cornea 20 as shown in FIG. 2 will have an anterior surface 22, a posterior surface 24 and a periphery 26 that interconnects the surfaces 22 and 24. Mathematically, the anterior surface 22 and the posterior surface 24 are both considered as being conic sections. For the generalized model 14, an axis 28 is defined that is perpendicular to the surfaces 22 and 24, and it passes through respective apexes 30 and 32 of the surfaces 22 and 24. Thus, as shown in FIG. 2, the curvatures of the anterior surface 22 and the posterior surface 24 are approximated by a respective conic section expressed as:

$$z ( x ) = \frac{1}{{e}^{2} - 1} [ \sqrt{{R}^{2} + {x}^{2} ( {e}^{2} - 1 )} - R ] .$$

In the above expression, the radius of curvature “R” for the anterior surface 22 is approximately 7.86 mm; the radius of curvature “R” for the posterior surface 24 is approximately 6.76 mm; and “e” for the eccentricity of the cornea 20 is 0.32. Collectively, this information is input to the computer 12 as mathematical approximations 18.

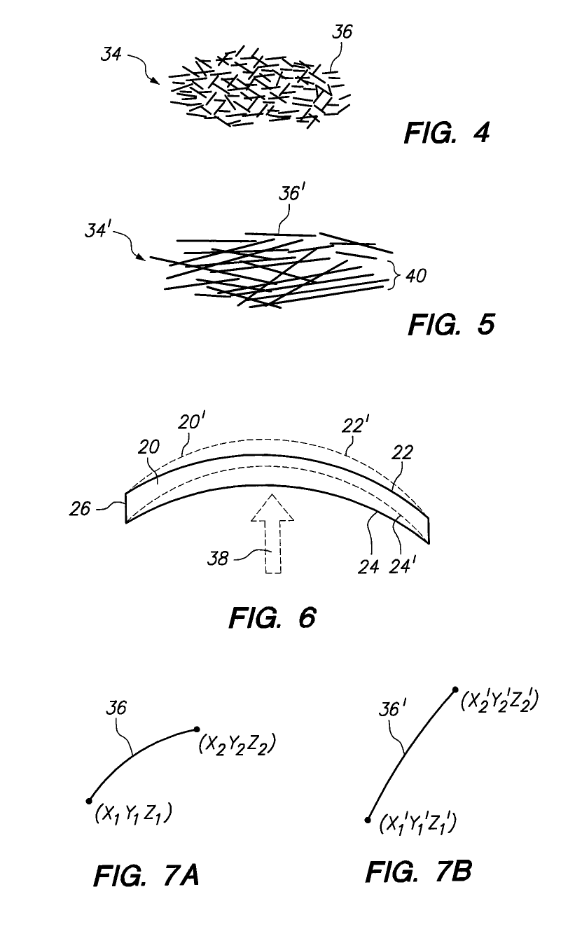

Corneal tissue between the anterior surface 22 and the posterior surface 24 consists of a plurality of collagen lamellae, such as the exemplary collagen lamellae 34a and 34b shown in FIG. 3. Within each lamella 34 there are a plurality of collagen fibers 36. And, the collagen fibers 36 will differ from each other, according to the nature of tissue that is involved. For example, with reference to FIG. 2, consider a lamella 34 located in Bowman's membrane of cornea 20. Also consider a lamella 34′ that is located in the stroma of cornea 20. In this example, the collagen fibers 36 of the lamella 34 (in Bowman's membrane) will be generally arranged as represented in FIG. 4. On the other hand, collagen fibers 36′ of the lamella 34′ (in the stroma) will be generally arranged as represented in FIG. 5. When comparing FIG. 4 with FIG. 5 it is to be appreciated that the collagen fibers 36 of lamella 34 shown in FIG. 4 are shorter, and have more linking bonds with other fibers 36. Further, they are more densely arranged than are the fibers 36′ in the lamella 34′ of the stroma shown in FIG. 5. In an accepted classification scheme, the fibers 36 in Bowman's membrane (FIG. 4) are classified as “type I or III.” On the other hand, fibers 36′ in the stroma (FIG. 5) are classified as either “type V” or “type VI”. Stated differently, with a higher number “type”, a collagen fiber 36 will be longer, have less cross linking bonds with other fibers 36, and will be less densely arranged. Importantly, these differences can be quantified.

Referring now to FIG. 6, a representative cross section of the cornea 20 is shown with a superposed cornea 20′ to demonstrate a change in configuration of the cornea 20 caused by a pressure differential (represented by the arrow 38). More specifically, the cornea 20 is shown responding to normal intraocular pressure in the eye. On the other hand, the cornea 20′ shows a response due to an increased pressure (i.e. pressure differential 38). The actual pressure differential 38 can be measured and imposed in accordance with known techniques. For purposes of the present invention, this pressure differential 38 affords the opportunity to obtain and evaluate additional information (i.e. mathematical characteristics) pertaining to collagen fibers 36 in the cornea 20. To do this, images of both the cornea 20 and the cornea 20′ are taken from the patient as disclosed above.

By cross referencing FIG. 6 with FIGS. 7A and 7B, the effect that a pressure differential 38 will have on individual collagen fibers 36 in the cornea 20 can be appreciated. For this comparison, the fiber 36 shown in FIG. 7A corresponds to the condition for cornea 20 shown in FIG. 6 (i.e. no pressure differential has yet been imposed on the cornea 20). In FIG. 7B, the fiber 36′ (i.e. the same fiber 36 as is shown in FIG. 7A) is shown after a pressure differential 38 has been imposed. As indicated above, the configuration of fiber 36 (FIG. 7A) and the configuration of fiber 36′ (FIG. 7B) can each be imaged. These images are then compared and the configuration changes of the fiber 36/36′ are measured. More specifically, the end coordinates (x₁y₁z₁ and x₂y₂z₂) of fiber 36 can be compared with the end coordinates (x′₁y′₁z′₁ and x′₂y′₂z′₂) of fiber 36′. This then provides information needed for calculating the mathematical characteristics that will identify the elasticity and viscosity of the fiber 36. Additionally, generally known information about the “type” of the fiber 36 (e.g. “type I or III”) can be used to further refine the mathematical characteristics of the fiber 36. Also, to facilitate programming the computer 12, it can happen that a group 40 of aligned fibers 36 can be identified (see FIG. 5). If so, each fiber 36 in the group 40 can be given the same mathematical characteristics. This may particularly be possible in the case of fibers 36 in the stroma where the fibers 36 are less dense and more likely to be aligned with other fibers 36.

As will be appreciated by the skilled artisan, the mathematical characteristics considered above can be ascertained for tens or hundreds of thousands of different fibers 36. Collectively, these mathematical characteristics are used to create the diagnostic data 16 that is input to the computer 12. This diagnostic data 16, together with the mathematical approximations 18 mentioned above that are used for configuring the anterior surface 22 and the posterior surface 24 of the cornea 20 establish and define the generalized model 14 for the system 10 of the present invention. Further, use of the diagnostic data 16 and the mathematical approximation 18 recognize that the resultant generalized model 14 is axisymmetric and is based on a nonlinearly elastic, slightly compressible, transversely isotropic formulation with an isotropic exponential Lagrangian strain-energy function based on:

W=½C(e^{Q}−1)+C_{compr}(I₃InI₃−I₃+1)

and

Q=b_{ff}E²_{ff}+bₓₓ(E²_{cc}+E²ₛₛ+E²_{cs}+E²_{sc})+b_{fx}(E²_{fc}+E²_{cf}+E²_{fs}+E²_{sf})

### Where:

I are invariants,

W is the strain potential (strain-energy function),

C is stress-scaling coefficient,

C_{compr} is bulk modulus (kPa),

E is strain,

b_{ff} is fiber strain exponent,

bₓₓ is transverse strain component, and

b_{fx} is fiber-transverse shear exponent.

For an operation of the system 10 of the present invention, the computer 12 is programmed to create the generalized model 14. To do this, the diagnostic data 16 and the mathematical approximations 18 are provided as input to the computer 12. Once the generalized model 14 has been created, selected elements in the model 14 can then be minimized to stimulate a surgical procedure. In effect, such a minimization of elements mimics a proposed cut, or a number of cuts in the cornea 20 (preferably the stroma). The response of the generalized model 14 can then be evaluated. And, based on the response, additional iterations of the process can be made if needed. In any event, the information obtained from operation of the generalized model 14 can be used for the preparation and conduct of an actual surgical procedure.

While the particular Generalized Modeling of the Cornea as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for simulating a reshaping of a model cornea which comprises:
a computer programmed with a generalized model comprising a plurality of elements, wherein each element is pre-programmed to simulate biomechanical characteristics of a collagen fiber in the cornea, and further wherein biomechanical characteristics of the pre-programmed elements are established based on diagnostic corneal data;
a first computer means connected to the generalized model for minimizing designated biomechanical characteristics on at least one selected element; and
a second computer means electronically connected to the generalized model for evaluating a reshaped model cornea in response to operation of the first computer means.

2. A system as recited in claim 1 wherein the generalized model defines an anterior surface and a posterior surface for the cornea, with an axis perpendicular to the surfaces and passing through respective apexes of the surfaces, and further wherein the curvatures of the anterior and posterior surfaces are approximated by a respective conic section.

3. A system as recited in claim 2 wherein the conic section for each surface is expressed as:
$$z ( x ) = \frac{1}{{e}^{2} - 1} [ \sqrt{{R}^{2} + {x}^{2} ( {e}^{2} - 1 )} - R ] .$$

4. A system as recited in claim 3 where:
R for the anterior surface is approximately 7.86 mm;
R for the posterior surface is approximately 6.76 mm; and
e for the eccentricity of the cornea is 0.32.

5. A system as recited in claim 1 wherein the first computer means minimizes an element by reducing its pre-programmed biomechanical characteristics approximately ninety percent in value.

6. A system as recited in claim 1 wherein each element includes information regarding shape, elasticity and viscosity of the collagen fiber.

7. A system as recited in claim 1 wherein the first computer means simulates a cut inside the stroma of the cornea, substantially parallel to the axis.

8. A system as recited in claim 1 wherein the first computer means simulates a cut inside the stroma, substantially perpendicular to the axis.

9. A system as recited in claim 1 wherein the generalized model is axisymmetric and is based on a nonlinearly elastic, slightly compressible, transversely isotropic formulation with an isotropic exponential Lagrangian strain-energy function based on:
W=½C(e^{Q}−1)+C_{compr}(I₃InI₃−I₃+1)
and
Q=b_{ff}E²_{ff}+bₓₓ(E²_{cc}+E²ₛₛ+E^{s}_{cs}+E²_{sc})+b_{fx}(E²_{fc}+E²_{cf}+E²_{fs}+E²_{sf})
Where:
I are invariants,
W is the strain potential (strain-energy function),
C is stress-scaling coefficient,
C_{compr} is bulk modulus (kPa),
E is strain,
b_{ff} is fiber strain exponent,
bₓₓ is transverse strain component, and
b_{fx} is fiber-transverse shear exponent.

10. A system for simulating a reshaping of a model cornea which comprises:
a generalized model having a plurality of individual elements, wherein each element is pre-programmed with biomechanical characteristics based on diagnostic corneal data pertinent to individual collagen fibers in the cornea to collectively replicate biomechanical properties of the cornea, and to represent the cornea in a first configuration;
a means connected to the generalized model for minimizing designated biomechanical characteristics on a plurality of selected elements; and
a means for evaluating a second configuration for the cornea in response to operation of the minimizing means.

11. A system as recited in claim 10 wherein the generalized model defines an anterior surface and a posterior surface for the cornea, with an axis perpendicular to the surfaces and passing through respective apexes of the surfaces, and further wherein the curvatures of the anterior and posterior surfaces are approximated by a respective conic section expressed as:
$$z ( x ) = \frac{1}{{e}^{2} - 1} [ \sqrt{{R}^{2} + {x}^{2} ( {e}^{2} - 1 )} - R ]$$
Where:
R for the anterior surface is approximately 7.86 mm;
R for the posterior surface is approximately 6.76 mm; and
e for the eccentricity of the cornea is 0.32.

12. A system as recited in claim 11 wherein the generalized model is axisymmetric and is based on a nonlinearly elastic, slightly compressible, transversely isotropic formulation with an isotropic exponential Lagrangian strain-energy function based on:
W=½C(e^{Q}−1)+C_{compr}(I₃InI₃−I₃=1)
and
Q=b_{ff}E²_{ff}+bₓₓ(E²_{cc}+E²ₛₛ+E²_{cs}+E²_{sc}) 30 b_{fx}(E²_{fc}+E²_{cf}+E²_{fs}+E²_{sf})
Where:
I are invariants,
W is the strain potential (strain-energy function),
C is stress-scaling coefficient,
C_{compr} is bulk modulus (kPa),
E is strain,
b_{ff} is fiber strain exponent,
bₓₓ is transverse strain component,
b_{fx} is fiber-transverse shear exponent, and
wherein the stress-scaling coefficient for Bowman's capsule (C_{Bowman}) is approximately five times greater than the stress-scaling coefficient for the stroma (Cₛₜᵣₒₘₐ).

13. A system as recited in claim 10 wherein elements are minimized by reducing pre-programmed biomechanical characteristics approximately ninety percent in value.

14. A system as recited in claim 10 wherein data for each collagen fiber is obtained from images of the cornea and includes information pertaining to the shape, elasticity and viscosity of each respective collagen fiber.

15. A method for simulating a reshaping of a cornea which comprises the steps of:
creating a generalized model comprising a plurality of elements;
pre-programming the plurality of elements to respectively simulate biomechanical characteristics of individual collagen fibers in the cornea, wherein the pre-programmed elements are established for the generalized model according to diagnostic corneal data;
minimizing biomechanical characteristics on selected elements;
measuring a reshaped cornea in response to the minimizing step; and
repeating the minimizing and measuring steps, as required.

16. A method as recited in claim 15 wherein the creating step further comprises the steps of:
imaging the cornea to obtain a first set of images of collagen fibers therein at a first pressure in the eye;
changing the first pressure in the eye to a second pressure in the eye;
imaging the cornea to obtain a second set of images of collagen fibers therein at the second pressure in the eye; and
comparing the first set of images with the second set of images to obtain the diagnostic corneal data.

17. A method as recited in claim 16 wherein the diagnostic corneal data includes information regarding the shape, elasticity and viscosity of individual collagen fibers in the cornea.

18. A method as recited in claim 15 wherein the generalized model defines an anterior surface and a posterior surface for the cornea, with an axis perpendicular to the surfaces and passing through respective apexes of the surfaces, and further wherein the curvatures of the anterior and posterior surfaces are approximated by a respective conic section expressed as:
$$z ( x ) = \frac{1}{{e}^{2} - 1} [ \sqrt{{R}^{2} + {x}^{2} ( {e}^{2} - 1 )} - R ] .$$
